(19) United States
(12) Patent Application Publication
(10) Pub. No.: US 2008/0220267 A1
(43) Pub. Date: Sep. 11, 2008

(54) Glyceride Compounds And Uses Thereof

(75) Inventors: James Alan Stephen Howell, Crewe (GB); Con Robert McElroy, Eire (GB); Robert Heathcote, Stoke-on-Trent (GB); Derek Peter Taylor, Shropshire (GB)

Correspondence Address: SWANSON & BRATSCHUN, L.L.C., 8210 SOUTHPARK TERRACE, LITTLETON, CO, 80120, US

(73) Assignees: KEELE UNIVERSITY, Keele, Staffordshire (GB); ADVANCE ENTERPRISES LTD, Wolverhampton (GB)

(21) Appl. No.: 11/997,003

(22) PCT Filed: Jul. 20, 2006
(86) PCT No.: PCT/GB06/02696

§ 371 Date: Jan. 28, 2008

(30) Foreign Application Priority Data
Aug. 2, 2005 (GB) 0515942.1

(51) Int. Cl. B32B 27/06; C08G 63/48; C07C 51/43

(52) U.S. Cl. 428/454; 528/295.5; 554/227; 554/213; 528/271; 428/704; 428/537.1

(57) ABSTRACT

A glyceride compound comprising a first aliphatic group and a first substituent group bonded to said first aliphatic group. The first substituent group has the formula —O(CH₂CH₂O)ₙP where n is 1 to 14 and P is a polymerisable group. An aqueous emulsion of the glyceride can be used in an emulsion polymerisation reaction to yield a polymer resin, which can then be mixed with a suitable base material, such as wood strands, to produce a composite material, such as Oriented Strand Board.

The present invention relates to glyceride compounds and uses thereof, particularly, but not exclusively, in the production of glyceride-based resins and composite materials comprising glyceride-based resins.

Composite materials such as Oriented Strand Board (OSB), Medium Density Fibreboard (MDF) and ceramic tiles are typically manufactured by blending a liquid resin with an appropriate raw material such as wood fibre or builders sand to form a deformable mass which is then moulded, often at elevated temperature, to form the final product.

A drawback of current methods for the manufacture of composite materials is that they commonly employ resins which are potentially harmful to the environment and/or humans. For example, formaldehyde-based resins are known to be environmentally harmful and formaldehyde has recently been classified as a human carcinogen. Moreover, resins used in the manufacture of composite materials are often produced by polymerising monomers derived from non-renewable sources in the presence of petroleum-based surfactants.

An object of the present invention is to obviate or mitigate one or more of the above drawbacks associated with current methods for the manufacture of composite materials.

According to an aspect of the present invention there is provided a glyceride compound for use in the production of a composite material, said glyceride compound comprising a first aliphatic group and a first substituent group bonded to said first aliphatic group, wherein the first substituent group has the formula —O(CH₂CH₂O)ₙP where n is 1 to 14 and P is a polymerisable group.

The present invention provides a family of glyceride compounds comprising aliphatic groups with substituent groups containing at least one ethylene oxide moiety and terminating with a polymerisable group. The number of ethylene oxide moieties can be chosen to suit a particular application. For example, substituent groups including a higher number of ethylene oxide moieties will generally be more water soluble than those containing fewer ethylene oxide moieties. Thus, the aqueous solubility of the glyceride compound can be tailored to suit a specific application or manufacturing process.

The ability to control the hydrophobicity of a glyceride compound enables the properties of the compound to be optimised for use in processes employing aqueous solvents or more polar solvents than glycerides that have not been modified in this way. For example, by combining one or more ethylene oxide moieties with a terminal polymerisable group, such as a methacrylate group, in the aliphatic substituent group, the glyceride compound can be copolymerised with other compatible reactive monomers under aqueous conditions.

The number of ethylene oxide moieties in the substituent group may be chosen to provide sufficient water compatibility to allow an aqueous emulsion of the glyceride to be formed which can then be used in an emulsion polymerisation reaction to yield a polymer resin. Such a resin can then be mixed with a suitable base material, such as wood strands, the resin crosslinked and the mixture pressed or moulded in to a composite material, such as Oriented Strand Board. Further aspects of the present invention provide a polymer resin incorporating glyceride monomers derived from the inventive glyceride compound, composite materials comprising such a polymer resin and methods for producing the same.

The ethylene oxide moiety may be considered a water solubilising group in that it confers a greater degree of aqueous solubility on the glyceride compound than the compound would possess if the ethylene oxide moiety were replaced with an ethylene moiety. The extent to which a given ethylene oxide substituent group modifies the aqueous solubility of a particular glyceride compound will depend, at least in part, upon the length of the aliphatic group to which the substituent group is bonded and the nature of the other chemical groups present in the glyceride molecule. For example, if the aliphatic group carrying the substituent group is relatively long and hydrophobic, e.g. 18 to 24 carbon atoms long, it is envisaged that the substituent group should contain a higher number of ethylene oxide moieties, e.g. 5 to 10 moieties, in order to improve the aqueous solubility of a glyceride compound containing these groups to the same extent as a substituent group incorporating fewer ethylene oxide moieties bonded to a shorter aliphatic group.

It will be appreciated that, particularly where substituent groups of longer chain length are provided, the glyceride compound may contain compounds incorporating substituent groups with a range of different chain lengths. In such cases, the quoted number of ethylene oxide moieties (e.g. 4 ethylene oxide moieties in polyethylene glycol 200 and 6 ethylene oxide moieties in polyethyleneglycol methacrylate 360) represents a calculated average value (rounded to the nearest integer) of the number of ethylene oxide repeating units present in the substituent groups of different lengths incorporated in to the glyceride compound.

A further related aspect of the present invention provides a method for producing a substituted glyceride compound comprising a first aliphatic group and a first substituent group bonded to said first aliphatic group in which the substituent group has the formula —O(CH₂CH₂O)ₙP where n is 1 to 14 and P is a polymerisable group, wherein the method comprises reacting a glyceride starting compound having an epoxide group bonded to an aliphatic group of the starting compound with a reactant compound having the formula HO(CH₂CH₂O)ₙP or a salt thereof so as to effect cleavage of the epoxide ring of the starting compound to provide said substituted glyceride compound.

The glyceride compound typically will be produced by epoxidising an unsaturated natural oil, such as a vegetable oil like rapeseed oil or palm oil, and then reacting the epoxidised glyceride compounds present in the oil with a nucleophilic reactant compound incorporating the substituent group to be added. The pattern of substitution within a particular oil will therefore be at least partly dependent upon the mixture of mono-, di- and tri-glycerides present in the oil and their pattern of unsaturation, since epoxidisation will occur at the sites of unsaturation present in each glyceride molecule.

Although the present invention contemplates the use of a monoglyceride compound, it is preferred that the glyceride compound is a diglyceride and thus comprises a second aliphatic group connected to a different carbon atom of the glycerol moiety than the carbon atom to which the first aliphatic group is connected.

In a preferred embodiment a second substituent group is bonded to the second aliphatic group and said second substituent group has the formula —O(CH₂CH₂O)ₘR¹, where m is to 14 and R¹ is selected from the group consisting of hydrogen, alkyl and a polymerisable group. Thus, for example, the second substituent group may be a hydroxyl group (where m is 0 and R¹ is hydrogen) or an alkoxide group (where m is 0 and R¹ is alkyl). The water compatibility of the glyceride molecule may again be controlled by the provision of one or more ethylene oxide groups in the second substituent group.

In a still further preferred embodiment the glyceride compound is a triglyceride compound in which case the glyceride compound comprises a third aliphatic group connected to the remaining carbon atom of the glycerol moiety. An example of a triglyceride is triolein 1, shown below. Preferably a third substituent group is bonded to the third aliphatic group and said third substituent group has the formula —O(CH₂CH₂O)_{q}R², where q is 0 to 14 and R² is selected from the group consisting of hydrogen, an alkyl group and a polymerisable group.

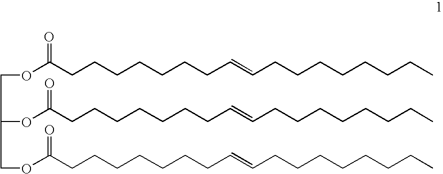

The number of ethylene oxide groups present in the first, second and/or third substituent group(s) may be selected to optimise the water compatibility of the glyceride compound to a particular application. While n may take any value in the range 1 to 14, n is preferably 2 to 12, more preferably n is 3 to 10, yet more preferably n is 4 to 8, and most preferably n is 5 to 6. It has been observed that for the compound triolein 1, which is the main triglyceride component of rapeseed oil, a satisfactory degree of water compatibility to permit subsequent emulsion polymerisation is achieved by attaching a substituent group to one of the aliphatic groups which contains approximately five ethylene oxide groups, i.e. in which n is approximately 5, and which comprises methacrylate as the polymerisable group (see compound 2 below).

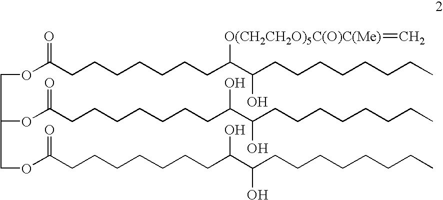

Since the first substituent group contains at least one ethylene oxide group, i.e. n is at least 1, the second and/or third substituent groups, when present, may contain between 0 and 14 ethylene oxide groups (i.e. m and/or q=0 to 14), more preferably 2 to 12, 3 to 10, 4 to 8, and most preferably 5 to 6 ethylene oxide groups.

It will also be appreciated that there may be one or more substituent groups bonded to each aliphatic group. Provided there is at least one first substituent group (i.e. a group having the formula O(CH₂CH₂O)ₙP where n is 1 to 14 and P is a polymerisable group) present in the glyceride compound the number and substitution pattern of any additional substituent groups present in the compound may be selected to suit a particular application.

In preferred embodiments of the present invention the or each polymerisable group, P, R¹, R² is a free radical addition polymerisable group, which may comprise a carbon-to-carbon double bond and may therefore be ethylenically unsaturated. Preferably the polymerisable group is derived from substituted or unsubstituted acrylic acid and most preferably the polymerisable group is derived from methacrylic acid, as shown above, by way of example, in compound 2.

Glycerides present in natural oils are typically esters of fatty acids and glycerol. The aliphatic group(s) in such natural glycerides will therefore be derived from fatty acids. It is preferable that the first aliphatic group in the glyceride compound contains 4 to 26 carbon atoms, more preferably 8 to 22 carbon atoms, still more preferably 10 to 20 carbon atoms and most preferably approximately 18 carbon atoms as in triolein. When present, the second and/or third aliphatic groups may contain 4 to 26 carbon atoms, more preferably 8 to 22 carbon atoms, still more preferably 10 to 20 carbon atoms and most preferably approximately 18 carbon atoms.

Although the aliphatic group may contain any desired degree of unsaturation, it is preferred that the first aliphatic group is saturated. In the case where the glyceride compound is derived from a natural source, the aliphatic group(s) originally present in each underivatised glyceride molecule will generally be unsaturated to some degree.

One way to introduce the substituent group into the glyceride molecule is to epoxidise sites of unsaturation in the glyceride compound and then react the resultant epoxide groups with a nucleophilic reactant containing the desired substituent group.

It will be evident that this method also provides a convenient means for providing one or more hydroxyl groups in each glyceride molecule, which may be further derivatised if desired. Preferably, a first hydroxyl group is bonded to the first aliphatic group. It is particularly preferred that the first hydroxyl group is bonded to the first aliphatic group at a position which is α to the position at which the first substituent group is bonded to the first aliphatic group, e.g. as shown above in compound 2.

In a further preferred embodiment of the present invention the glyceride compound has the formula

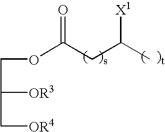

where X¹ is the first substituent group having the formula —O(CH₂CH₂O)ₙP, n is 1 to 14 and P is a polymerisable group, s is 0 to 23, t is 1 to 24, s+t=2 to 24, and R³ and R⁴ are each separately selected from the group consisting of hydrogen, a saturated or unsaturated aliphatic group, such as an alkyl group (e.g. methyl, ethyl, propyl etc) or an alkylene group (e.g. ethylene, propylene etc), and a group having the formula

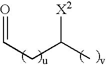

where X² is —O(CH₂CH₂O)_{w}R⁵, w is 0 to 14, R⁵ is hydrogen, an alkyl group or a polymerisable group, u is 0 to 23, v is 1 to 24 and u+v=2 to 24.

As defined above, n is selected from the range 1 to 14 and P is the polymerisable group hereinbefore described. The value for n may be chosen to confer the required degree of water compatibility to the glyceride compound, and as defined above, n is preferably 2 to 12, 3 to 10, 4 to 8, and most preferably n is 5 to 6. While s may be 0 to 23, s is preferably 1 to 14, more preferably 4 to 10 and most preferably around 8. The value of t may be selected from the range 1 to 24, however, it is preferred that t is 2 to 14, more preferably 4 to 12 and most preferably around 9. The values of s and t are subject to the proviso that the sum of s and t lies in the range 2 to 24.

In respect of R³ and R⁴, w is preferably 2 to 12, 3 to 10, 4 to 8, and most preferably 5 to 6. The polymerisable group, R⁵, is preferably a free radical addition polymerisable group, which may comprise a carbon-to-carbon double bond. Preferably R⁵ is derived from substituted or unsubstituted acrylic acid and most preferably the polymerisable group is derived from methacrylic acid. Although u may be 0 to 23, u is preferably 1 to 14, more preferably 4 to 10 and most preferably around 8. The value of v may be selected from the range 1 to 24, however, it is preferred that v is 2 to 14, more preferably 4 to 12 and most preferably around 9. The values of u and v are subject to the proviso that the sum of u and v lies in the range 2 to 24.

According to a further preferred embodiment of the present invention the glyceride compound preferably has the formula

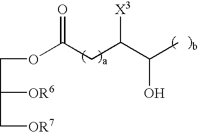

where X³ is the first substituent group having the formula —O(CH₂CH₂O)ₙP, n is 1 to 14 and P is a polymerisable group, a is 0 to 22, b is 1 to 23, s+t=1 to 23, and R¹ and R⁷ are each separately selected from the group consisting of hydrogen, a saturated or unsaturated aliphatic group and a group having the formula

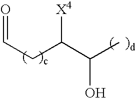

where X⁴ is —O(CH₂CH₂O)_{y}R⁸, y is 0 to 14, R⁸ is hydrogen, an alkyl group or a polymerisable group, c is 0 to 22, d is 1 to 23 and c+d=1 to 23.

The definitions of n and P are again as hereinbefore described, i.e. n is 1 to 14 and P is the polymerisable group as defined above. The value for n is preferably 2 to 12, 3 to 10, 4 to 8, and most preferably n is 5 to 6.

While a may be 0 to 22 and b may be 1 to 23, at least one of a and b is preferably 1 to 14, more preferably 4 to 10 and most preferably around 8. The values of a and b are subject to the proviso that the sum of a and b lies in the range 1 to 23.

With respect to R⁶ and R⁷, y is preferably 2 to 12, 3 to 10, 4 to 8, and most preferably 5 to 6. R⁸ is preferably a free radical addition polymerisable group, which may comprise a carbon-to-carbon double bond.

Preferably R⁸ is derived from substituted or unsubstituted acrylic acid and most preferably R¹ is derived from methacrylic acid. Although c may be 0 to 22 and d may be 1 to 23, at least one of c and d is preferably 1 to 14, more preferably 4 to 10 and most preferably around 8.

As stated above, a further aspect of the present invention provides a method for producing a substituted glyceride compound (e.g. compound 2) by reacting a glyceride starting compound having an epoxide group bonded to an aliphatic group of the starting compound (e.g. epoxidised triolein 3 below) with a reactant compound (e.g. HO(CH₂CH₂O)₅C(O)C(Me)═CH₂) to effect cleavage of the epoxide ring of the starting compound to provide said substituted glyceride compound.

In the example shown below, the pattern of substitution may be achieved by reacting the epoxidised glyceride compound with the reactant compound in the molar ratio of approximately 3:1 (glyceride starting compound:reactant compound).

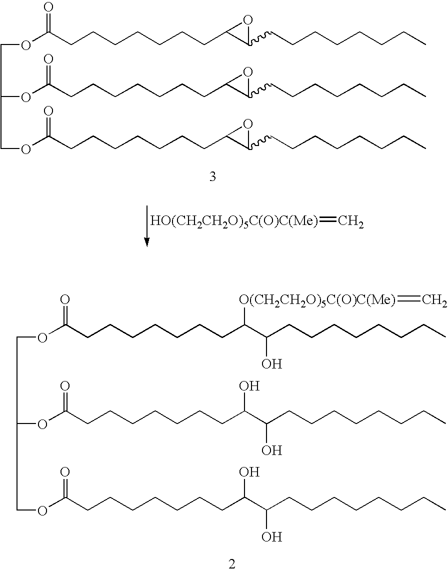

It is preferred that cleavage of the epoxide ring is effected in the presence of a Lewis acid catalyst, which may comprise boron trifluoride, and is preferably boron trifluoride etherate.

The glyceride starting compound, reactant compound and catalyst may be combined in any appropriate order but in one preferred embodiment a solution of the reactant compound and the catalyst is formed prior to reacting the glyceride starting compound with the reactant compound.

In a further preferred embodiment the glyceride starting compound and the reactant compound are dissolved in a first organic solvent prior to addition of the catalyst. Conveniently, the catalyst may be provided as a solution in a second organic solvent. At least one of the first and second solvents is preferably diethyl ether.

The reacted glyceride compound incorporating the substituent group may be isolated in any convenient fashion, although it is preferred that the substituted glyceride compound is separated from the catalyst by adding water to induce a phase separation process whereby the substituted glyceride compound resides substantially in an organic phase and the catalyst resides substantially in an aqueous phase, followed by separation of the organic and aqueous phases.

It is preferred that the glyceride starting compound is comprised in a vegetable oil, such as rapeseed oil, linseed oil or palm oil, although the glyceride starting compound may be derived from any convenient natural or synthetic source.

The reactant compound should be chosen such that it will react with the epoxide group(s) present in the glyceride starting compound to provide the desired substituent group. One example of a particularly preferred reactant compound is polyethyleneglycol methacrylate. More than one type of reactant compound may be used. Thus, in a further preferred embodiment the method comprises the addition of a further reactant compound having the formula HO(CH₂CH₂O)ₙH where e is 1 to 14.

The conditions under which the epoxide ring opening reaction takes place should be chosen to best suit the chemical structure of the glyceride starting compound and the reactant compound(s). In order to make the method particularly suitable for industrial application it is preferred that cleavage of the epoxide ring is effected at room temperature. Moreover, the substituted glyceride compound is preferably dispersed in water to provide an aqueous emulsion of the substituted glyceride, optionally in the presence of a polymerisation inhibitor, such as a substituted phenol compound.

It will be appreciated that the above described inventive method may be used to produce a substituted glyceride compound in accordance with the glyceride compound forming another aspect of the present invention hereinbefore described which is suitable for use in the production of a composite material.

A further aspect of the present invention provides a polymer resin for use in the production of a composite material, said resin being formed by copolymerising glyceride monomers and monomers of at least one further polymerisable compound, each glyceride monomer comprising a first aliphatic group and a first substituent group bonded to said first aliphatic group, wherein the first substituent group has the formula —O(CH₂CH₂O)ₙP, where n is 1 to 14 and P is a polymerisable group.

A yet further related aspect of the present invention provides a method for producing a polymer resin comprising copolymerising glyceride monomers and monomers of at least one further polymerisable compound, each glyceride monomer comprising a first aliphatic group and a first substituent group bonded to said first aliphatic group, wherein the first substituent group has the formula —O(CH₂CH₂O)ᵣP, where n is 1 to 14 and P is a polymerisable group.

The glyceride compound of the present invention may therefore be utilised to provide glyceride monomers for incorporation into a polymer resin, which can then be used to produce a composite material. Accordingly, n and P are as defined above in respect of the glyceride compound of the present invention, i.e. n is preferably 2 to 12, 3 to 10, 4 to 8, or most preferably 5 to 6, and P is a free radical addition polymerisable group which may be ethylenically unsaturated and is preferably derived from substituted or unsubstituted acrylic acid, such as methacrylic acid.

In a preferred embodiment the at least one further polymerisable monomer is polymerisable by free radical addition. Preferably the at least one further polymerisable monomer is ethylenically unsaturated and may be a substituted or unsubstituted acrylate compound, or a substituted or unsubstituted aromatic compound. The resin is preferably formed by copolymerising said glyceride monomers with monomers of a substituted or unsubstituted acrylate compound, such as acrylic acid, and monomers of a substituted or unsubstituted aromatic compound, such as styrene.

In a preferred embodiment of the method to produce the polymer resin the glyceride monomers to be copolymerised are provided in the form of an emulsion, which preferably comprises the glyceride monomers dispersed in an aqueous medium.

In order to improve the efficiency of the emulsion polymerisation process the copolymerisation reaction is effected in the presence of a polymerisation initiator, for example, a free radical polymerisation initiator. Moreover, the copolymerisation reaction is preferably effected at a temperature in the range 20 to 100° C., more preferably 40 to 90° C., and most preferably at a temperature of approximately 80° C.

As mentioned above it is preferred that the polymer resin is formed by copolymerising said glyceride monomers with monomers of different first and second polymerisable compounds. The monomers of the first and second polymerisable compounds are preferably mixed prior to reaction with the glyceride monomers.

According to another aspect of the present invention there is provided a composite material comprising a base material and a crosslinked polymer resin formed by copolymerising glyceride monomers and monomers of at least one further polymerisable compound to form a polymer resin and crosslinking the polymer resin, each glyceride monomer comprising a first aliphatic group and a first substituent group bonded to said first aliphatic group, wherein the first substituent group has the formula —O(CH₂CH₂O)ₙP, where n is 1 to 14 and P is a polymerisable group.

Another related aspect of the present invention provides a method for producing a composite material comprising a base material and a crosslinked polymer resin, the method comprising copolymerising glyceride monomers and monomers of at least one further polymerisable compound to provide a polymer resin, mixing the base material and polymer resin, crosslinking the polymer resin, and forming the base material and resin mixture into the composite material, each glyceride monomer comprising a first aliphatic group and a first substituent group bonded to said first aliphatic group, wherein the first substituent group has the formula —O(CH₂CH₂O)ₙP, where n is 1 to 14 and P is a polymerisable group.

The polymer resin of the present invention may therefore be used to produce a composite material in accordance with another aspect of the present invention. The glyceride monomers incorporated into the polymer resin are in accordance with the glyceride compound of the present invention. Particularly, in the first substituent group, n and P are as defined above in respect of the glyceride compound of the present invention, i.e. n is preferably 2 to 12, 3 to 10, 4 to 8, or most preferably 5 to 6, and P is a free radical addition polymerisable group which may be ethylenically unsaturated and is preferably derived from substituted or unsubstituted acrylic acid, such as methacrylic acid.

In a preferred embodiment the base material is an organic material or a ceramic material. The organic material may be selected from the group consisting of wood fibre, straw, and particulate or fibrous biomass material. The ceramic material is preferably sand but also may be any form of particulate quarry waste.

The method for producing a composite material preferably further comprises blending the polymer resin with a thermally activated crosslinking agent prior to mixing the polymer resin with the base material. It is preferred that an emulsion of the polymer resin is blended with the crosslinking agent at a ratio of up to 10% of the solids content of the polymer resin emulsion. More preferably an emulsion of the polymer resin is blended with the crosslinking agent at a ratio of up to 5% of the solids content of the polymer resin emulsion, and most preferably an emulsion of the polymer resin is blended with the crosslinking agent at a ratio of 2 to 3% of the solids content of the polymer resin emulsion.

Preferably crosslinking of the polymer resin is effected by increasing the temperature of the base material and resin mixture to a temperature which is sufficient to activate the crosslinking agent. Although the crosslinking agent may be selected so as to possess any appropriate activation temperature, it is preferred that the crosslinking agent has an activation temperature in the range 130° C. to 200° C., more preferably 140° C. to 190° C., and most preferably an activation temperature of approximately 150° C.

Any appropriate crosslinking agent may be used depending at least in part on the crosslinkable group(s) present in the polymer resin. For example, where each polymer molecule of the resin comprises at least one hydroxyl group, the crosslinking agent may effect crosslinking of the polymer resin by reacting with the hydroxyl groups present in the polymer resin. The hydroxyl groups may, for example, be present in the resin as a result of the glyceride monomer, which is incorporated into the polymer resin, being formed by reacting an epoxidised glyceride compound (e.g. epoxidised triolein 3 described above) with a reactant compound (e.g. HO(CH₂CH₂O)₅C(O)C(Me)═CH₂) which cleaves the epoxide ring(s) of the epoxidised glyceride compound and provides one or more hydroxyl groups (see compound 2 above). A particularly preferred crosslinking agent which reacts with hydroxyl groups in the polymer resin is an epoxy prepolymer, such as a diglycidyl prepolymer obtained from Schill and Seilacher Limited, UK.

Where each polymer molecule of the resin includes one or more carboxylic acid groups the cross linking agent preferably effects crosslinking of the polymer resin by reacting with the carboxylic acid groups present in the polymer resin. The carboxylic acid groups may be present as a result of copolymerising the glyceride monomers with monomers which contain carboxylic acid groups, such as acrylic acid. A preferred crosslinking agent which reacts with carboxylic acid groups present in the polymer resin is Beetle resin PT336 melamine-formaldehyde obtained from BIP Limited, UK.

In a preferred embodiment the base material and the polymer resin are mixed to provide a resin loading of 5% to 25% by dry weight of the base material, more preferably these components are mixed to provide a resin loading of approximately 10% by dry weight of the base material.

The base material and polymer resin mixture is preferably dried to remove excess moisture prior to crosslinking of the polymer resin. It is preferred that, after drying, the base material and polymer resin mixture has a water content of less than around 10%, more preferably less than around 6% and most preferably less than around 3% by weight.

Any convenient method may be used to process the composite material into its final form. Preferably the base material and polymer resin mixture is formed into the composite material by moulding, extruding and/or pressing the mixture. When a thermally activated crosslinking agent is used, a particularly convenient stage in production of the composite material at which to activate the crosslinking agent is during forming of the final composite material, thus forming of the mixture into the composite material is preferably effected at a temperature which is sufficient to activate the crosslinking agent.

A further aspect of the present invention provides a glyceride compound for use as a surface active agent, said glyceride compound comprising a first aliphatic group and a first substituent group bonded to said first aliphatic group, wherein the first substituent group has the formula —O(CH₂CH₂O)_{f}Z where f is 1 to 14 and Z is selected from the group consisting of hydrogen, an alkyl group, a sulphite group and a phosphite group.

The combination in a single compound of a relatively hydrophobic aliphatic group with a substituent group incorporating a water solubilising moiety and an ionisable terminal moiety, such as a hydroxyl group, alkoxy group, sulphate group or phosphate group, enables glyceride compounds in accordance with this aspect of the present invention to be utilised as surfactants. The alkyl group is preferably a methyl group or an ethyl group.

In a preferred embodiment of this aspect of the present invention f is 2 to 12, more preferably 3 to 10, more preferably 4 to 8, and most preferably 5 to 6.

With respect to this aspect of the present invention the first aliphatic group preferably contains 4 to 26 carbon atoms, more preferably 8 to 22 carbon atoms, still more preferably 10 to 20 carbon atoms and most preferably approximately 18 carbon atoms. The first aliphatic group may possess any desired degree of unsaturation, although it is preferred that the first aliphatic group is saturated.

Preferably a first hydroxyl group is bonded to the first aliphatic group. The first hydroxyl group may be bonded to the first aliphatic group at a position which is α to the position at which the first substituent group is bonded to the first aliphatic group.

This aspect of the present invention contemplates surfactant compounds derived from monoglycerides containing a single aliphatic group but also di- and tri-glycerides. Thus, the surfactant glyceride compound preferably comprises a second aliphatic group. It is preferred that a second substituent group is bonded to the second aliphatic group and said second substituent group has the formula —O(CH₂CH₂O)_{g}Z¹ where g is 0 to 14 and Z¹ is selected from the group consisting of hydrogen, an alkyl group, a sulphite group and a phosphite group.

The number of ethylene oxide groups, g, may be 2 to 12, more preferably 3 to 10, more preferably 4 to 8, and most preferably 5 to 6.

Furthermore, the surfactant glyceride compound may comprise a third aliphatic group in which case there may be provided a third substituent group bonded to the third aliphatic group, wherein said third substituent group has the formula —O(CH₂CH₂O)ₕZ², where h is 0 to 14 and Z² is selected from the group consisting of hydrogen, an alkyl group (e.g. a methyl group or an ethyl group), a sulphite group and a phosphite group.

The number of ethylene oxide groups, h, may be 2 to 12, more preferably 3 to 10, more preferably 4 to 8, and most preferably 5 to 6.

At least one of the second and third aliphatic groups may contain 4 to 26 carbon atoms, more preferably 8 to 22 carbon atoms, still more preferably 10 to 20 carbon atoms and most preferably approximately 18 carbon atoms.

The glyceride compound according to this aspect of the present invention which is suitable for use as a surface active agent preferably has the formula

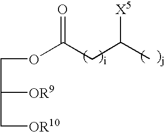

where X⁵ is the first substituent group having the formula —O(CH₂CH₂O)_{f}Z, f is 1 to 14 and Z is hydrogen, an alkyl group, a sulphite group or a phosphite group, i is 0 to 23, j is 1 to 24, i+j=2 to 24, and R⁹ and R¹⁰ are each separately selected from the group consisting of hydrogen, a saturated or unsaturated aliphatic group, such as an alkyl group (e.g. methyl, ethyl, propyl etc) or an alkylene group (e.g. ethylene, propylene etc), and a group having the formula

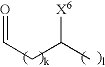

where X⁶ is —O(CH₂CH₂O)ₒR¹¹, o is 0 to 14, R¹¹ is hydrogen, an alkyl group, a sulphite group or a phosphite group, k is 0 to 23, l is 1 to 24 and k+1=2 to 24.

The value for f may be chosen to confer the required degree of water compatibility to the glyceride compound, and as defined above, f is preferably 2 to 12, 3 to 10, 4 to 8, and most preferably f is 5 to 6. While i may be 0 to 23, i is preferably 1 to 14, more preferably 4 to 10 and most preferably around 8. The value of j may be selected from the range 1 to 24, however, it is preferred that j is 2 to 14, more preferably 4 to 12 and most preferably around 9. The values of i and j are subject to the proviso that the sum of and j lies in the range 2 to 24. In respect of R⁹ and R¹⁰, o is preferably 2 to 12, 3 to 10, 4 to 8, and most preferably 5 to 6. Although k may be 0 to 23, k is preferably 1 to 14, more preferably 4 to 10 and most preferably around 8. The value of l may be selected from the range 1 to 24, however, it is preferred that 1 is 2 to 14, more preferably 4 to 12 and most preferably around 9. The values of k and l are subject to the proviso that the sum of k and l lies in the range 2 to 24.

A further preferred embodiment of the present invention provides a glyceride compound for use as a surface active agent having the formula

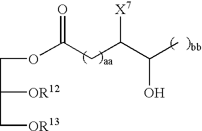

where X⁷ is the first substituent group having the formula —O(CH₂CH₂O)_{f}Z, f is 1 to 14 and Z is hydrogen, an alkyl group, a sulphite group or a phosphite group, aa is 0 to 22, bb is 1 to 23, aa+bb=1 to 23, and R¹² and R¹³ are each separately selected from the group consisting of hydrogen, a saturated or unsaturated aliphatic group, such as an alkyl group (e.g. methyl, ethyl, propyl etc) or an alkylene group (e.g. ethylene, propylene etc), and a group having the formula

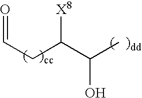

where X⁸ is —O(CH₂CH₂O)_{yy}R¹⁴, yy is 0 to 14, R¹⁴ is hydrogen, an alkyl group, a sulphite group or a phosphite group, cc is 0 to 22, dd is 1 to 23 and cc+dd=1 to 23.

The value for f is preferably 2 to 12, 3 to 10, 4 to 8, and most preferably f is 5 to 6. While aa may be 0 to 23, aa is preferably 1 to 14, more preferably 4 to 10 and most preferably around 8. The value of bb may be selected from the range 1 to 24, however, it is preferred that bb is 2 to 14, more preferably 4 to 12 and most preferably around 9. The values of aa and bb are subject to the proviso that the sum of aa and bb lies in the range 2 to 24.

In respect of R¹² and R¹³, yy is preferably 2 to 12, 3 to 10, 4 to 8, and most preferably 5 to 6. Although cc may be 0 to 22 and dd may be 1 to 23, at least one of cc and dd is preferably 1 to 14, more preferably 4 to 10 and most preferably around 8.

A related aspect of the present invention provides a method for producing a substituted glyceride compound for use as a surface active agent comprising a first aliphatic group and a first substituent group bonded to said first aliphatic group in which the substituent group has the formula —O(CH₂CH₂O)_{f}Z where f is 1 to 14 and Z is selected from the group consisting of hydrogen, an alkyl group, a sulphite group and a phosphite group, wherein the method comprises reacting a glyceride starting compound having an epoxide group bonded to an aliphatic group of the starting compound with a reactant compound having the formula HO(CH₂CH₂O)_{f}Z or a salt thereof so as to effect cleavage of the epoxide ring of the starting compound to provide said substituted glyceride compound.

The reactant compound HO(CH₂CH₂O)_{f}Z (e.g. HO(CH₂CH₂O)_{4average}H, as used in Example 10 below) is chosen to provide the desired number of water solubilising ethylene oxide groups attached to the or each aliphatic group, i.e. f may be 2 to 12, 3 to 10, 4 to 8, and most preferably f is 5 to 6.

The oxygen atom of one or the terminal hydroxide groups of the reactant compound acts as a nucleophile and attacks one of the epoxidised carbon atoms of the starting compound which results in cleavage of the or each epoxide ring of the starting compound to provide the substituted glyceride surfactant compound.

In the example shown below, the pattern of substitution may be achieved by reacting the epoxidised glyceride compound 3 with the reactant compound HO(CH₂CH₂O)_{4average}H in the molar ratio of approximately 3:1 (glyceride starting compound:reactant compound).

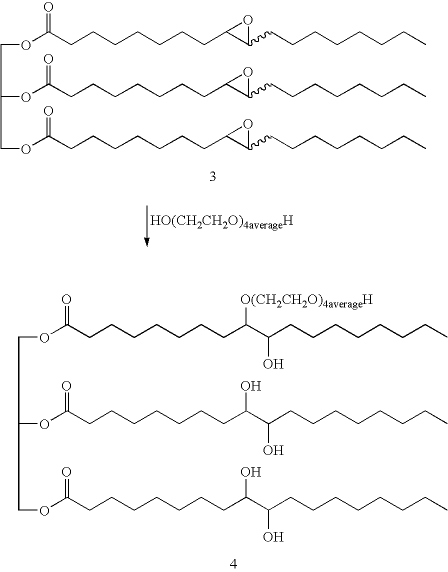

It is preferred that cleavage of the epoxide ring is effected in the presence of a Lewis acid catalyst, which may comprise boron trifluoride, and is preferably boron trifluoride etherate.

The glyceride starting compound, reactant compound and catalyst may be combined in any appropriate order but in one preferred embodiment a solution of the reactant compound and the catalyst is formed prior to reacting the glyceride starting compound with the reactant compound.

The reacted glyceride compound incorporating the substituent group may be isolated in any convenient fashion, although it is preferred that the glyceride compound is isolated by adding water followed by extraction with an organic solvent, such as dichloromethane, which can then be evaporated off to yield the glyceride surfactant compound.

It is preferred that the glyceride starting compound is comprised in a vegetable oil, such as rapeseed oil, linseed oil or palm oil, although the glyceride starting compound may be derived from any convenient natural or synthetic source.

As mentioned above, the reactant compound should be chosen such that it will react with the epoxide group(s) present in the glyceride starting compound to provide the desired substituent group. One example of a particularly preferred reactant compound is polyethyleneglycol. More than one type of reactant compound may be used. Thus, in a further preferred embodiment the method comprises the addition of a further reactant compound having the same general formula as the initial reactant compound (formula HO(CH₂CH₂O)_{f}Z) but which may contain a different number of ethylene oxide groups. The further reactant compound may therefore be represented by the formula HO(CH₂CH₂O)ₑₑH where ee is 1 to 14. As in the initial reactant compound, the number of ethylene oxide groups present in the further reactant compound may be 2 to 12, 3 to 10, 4 to 8, and most preferably ee is 5 to 6.

The conditions under which the epoxide ring opening reaction takes place should be chosen to best suit the chemical structure of the glyceride starting compound and the reactant compound(s). In order to make the method particularly suitable for industrial application it is preferred that cleavage of the epoxide ring is effected at room temperature.

### EXAMPLES

Example 1 below demonstrates a method for producing a modified rapeseed oil triglyceride in accordance with an aspect of the present invention. Example 2 sets out a method for producing a copolymer resin incorporating the triglyceride prepared in Example 1 which represents a further aspect of the present invention. Examples 3 and 4 show how the resin prepared in Example 2 may be used to produce ceramic and wood composite materials according to a further aspect of the present invention.

Examples 5 and 6 describe methods for producing modified rapeseed oil triglycerides in accordance with an aspect of the present invention. Example 7 sets out a method for producing a copolymer resin incorporating the triglyceride prepared in Example 5 which represents a further aspect of the present invention. Examples 8 and 9 demonstrate how the resin prepared in Example 7 may be used to produce ceramic and wood composite materials according to a further aspect of the present invention.

Example 10 provides a method for preparing a glyceride compound suitable for use as a surfactant in accordance with a further aspect of the present invention.

The modified rapeseed oil triglycerides mentioned below were all prepared from epoxidised rapeseed oil, which was prepared from rapeseed oil using the epoxidation procedure described in ‘Synthetic Methods’ (see infra).

The crosslinking agent used in the preparation of the composite materials in Examples 3, 4, 8 and 9 was a diglycidyl prepolymer obtained from Schill and Seilacher Limited, UK.

### Example 1

### Preparation of a Modified Rapeseed Oil Triglyceride

A solution of boron trifluoride etherate (4.0 g) was added to polyethyleneglycol methacrylate 360 (157 g) (average number of ethylene oxide groups per polymer chain=6) with stirring at room temperature. This solution was then transferred to a dropping funnel and added dropwise to a stirred flask containing epoxidised rapeseed oil (400 g) prepared as described in ‘Synthetic Methods’ (see infra). After completion of the reaction, the crude product (557 g) was dispersed in water (3000 ml) to produce a monomer emulsion which was used directly in the emulsion polymerisation procedure set out below in Example 2.

### Example 2

### Preparation of a Modified Rapeseed Oil Triglyceride-Based Resin

An emulsion polymerisation reaction was carried out in a 5 litre baffled jacketed reactor equipped with a PTFE anchor stirring blade operating at 400 rpm. The monomer emulsion from Example 1 was heated to 80° C. with vigorous stirring. A premixed solution of acrylic acid (128 g) in styrene (845 g) and a solution of potassium persulphate (23.6 g) in water (450 ml) were added simultaneously through separate feed streams over a period of 45 minutes. Efficient cooling was necessary to control the initially strong exotherm. The reaction was complete in approximately 3 hours (yield: 4970 g).

### Example 3

### Production of a Ceramic Composite Material

An emulsion polymer produced in accordance with Example 2 was blended with an epoxy crosslinking agent at a ratio of 1% of the solids content of the polymer. This mixture was then used to coat builders sand (500 g per tile, particle size: 90-180 μm) through a combination of agitation and spraying to give a loading of 10% by dry weight. The material was air dried until the water content was reduced to less than 3% by weight. The dried material was packed into mild steel moulds (150 mm×150 mm×10 mm) and pressed at 160-180° C. on a Bradley & Turton press (press capacity—100 tons on 45 cm diameter ram with 50 cm² platens with an approximate pressure of 39 bar) for 5 minutes. The tiles were removed from the moulds after cooling.

### Example 4

### Production of a Wood Composite Material

An emulsion polymer produced in accordance with Example 2 was blended with an epoxy crosslinking agent at a ratio of 1% of the solids content of the polymer. This mixture was then used to coat the wood strands (450-490 g) through a combination of agitation and spraying to give a loading by dry weight of 10%. The coated wood strands were then air dried to a water content below 3%. The dried material was packed into a Caul box (300 mm×300 mm) which in turn was placed on a woven steel Caul screen. This was pressed at 160-180° C. on a Bradley & Turton press (press capacity—100 tons on 45 cm diameter ram with 50 cm² platens with an approximate pressure of 39 bar) for 5 minutes to produce a board of 9 mm thickness which was removed from the Caul box after cooling.

### Example 5

### Preparation of a Modified Rapeseed Oil Triglyceride

Epoxidised rapeseed oil (1050 g) and polyethyleneglycol methacrylate 360 (405 g) (average number of ethylene oxide groups per polymer chain=6) were dissolved in diethyl ether (1750 ml) and stirred vigorously. A solution of boron trifluoride etherate (5 g) in diethyl ether (250 ml) was added dropwise and the reaction was stirred at room temperature until complete (approximately 3 hours). Water (100 ml) was added with vigorous stirring and the aqueous layer was separated to remove excess catalyst. The diethyl ether was removed under reduced pressure to give the crude product (yield: 1450 g) which was dispersed in water (2900 ml) to provide a 33% solids monomer emulsion (yield: 4350 g). 4-Ethoxyphenol was added at the 100 ppm level as a polymerisation inhibitor.

### Example 6

### Preparation of a Modified Rapeseed Oil Triglyceride

Epoxidised rapeseed oil (930 g), polyethyleneglycol methacrylate 360 (360 g) (average number of ethylene oxide groups per polymer chain=6) and polyethyleneglycol 200 (400 g) (average number of ethylene oxide groups per polymer chain=4) were dissolved in diethyl ether (1750 ml) and stirred vigorously. A solution of boron trifluoride etherate (5 g) in diethyl ether (250 ml) was added dropwise and the reaction was stirred at room temperature until complete (approximately 3 hours). Water (100 ml) was added with vigorous stirring and the aqueous layer was separated to remove excess catalyst. The diethyl ether was removed under reduced pressure to give the crude product (yield: 1690 g). The product was dispersed in water (3380 ml) to provide a 33% solids emulsion (yield 5070 g). 4-Ethoxyphenol was added at the 100 ppm level as a polymerisation inhibitor.

### Example 7

### Preparation of a Modified Rapeseed Oil Triglyceride-Based Resin

An emulsion polymerisation reaction was carried out in a 5 litre baffled jacketed reactor equipped with a PTFE anchor stirring blade operating at 400 rpm. The 33% solids dispersion of the monomer produced in accordance with Example 5 (1675 g) and water (1825 ml) were heated to 80° C. with vigorous stirring. A premixed solution of acrylic acid (128 g) in styrene (845 g) and a solution of potassium persulphate (23.6 g) in water (475 ml) were added simultaneously through separate feed streams over a period of 45 minutes. Efficient cooling was necessary to control the initially strong exotherm. The reaction was complete in approximately 3 hours (yield: 4970 g, viscosity: 40 cps).

### Example 8

### Production of a Ceramic Composite Material

An emulsion polymer produced in accordance with Example 7 was blended with an epoxy crosslinking agent at a ratio of 2-3% of the solids content of the polymer. This mixture was then used to coat builders sand (450 g, particle size: 90-180 μm) through a combination of agitation and spraying to give a loading of 10% by dry weight. The material was air dried until the water content was reduced to less than 3% by weight. The dried material was packed into mild steel moulds (150 mm×150 mm×10 mm) and pressed at 160-180° C. on a Bradley & Turton press (press capacity—100 tons on 45 cm diameter ram with 50 cm² platens with an approximate pressure of 39 bar) for 5 minutes. The tiles were removed from the moulds after cooling.

### Example 9

### Production of a Wood Composite Material

The wood strands used to produce the orientated strand board were produced from spruce/pine using a knife ring flaker. An emulsion polymer produced in accordance with Example 7 was blended with an epoxy crosslinking agent at a ratio of 2-3% of the solids content of the polymer. This mixture was then used to coat the wood strands (450-490 g) through a combination of agitation and spraying to give a loading by dry weight of 10%. The coated wood strands were then air dried to a water content below 3% and packed into a Caul box (300 mm×300 mm). This in turn was placed on a woven steel Caul screen. This was pressed at 160-180° C. on a Bradley & Turton press (press capacity—100 tons on 45 cm diameter ram with 50 cm² platens with an approximate pressure of 39 bar) for 5 minutes to produce a board of 9 mm thickness which was removed from the Caul box after cooling.

### Example 10

### Preparation of a Glyceride Surfactant Compound

A solution of boron trifluoride etherate (4.0 g) was added to polyethylene glycol 200 (172 g) (average number of ethylene oxide groups per polymer chain=4) with stirring at room temperature. This solution was then transferred to a dropping funnel and added dropwise to a stirred flask containing epoxidised rapeseed oil (400 g) prepared as described in ‘Synthetic Methods’ (see infra). After completion of the reaction, distilled water (1000 g) was added and the product was extracted with dichloromethane (250 ml). Evaporation of solvent gave the glyceride surfactant (540 g).

The product has a high solubility in water and can maintain as homogeneous a 1:1 mixture of toluene and water.

### Synthetic Methods

### Preparation of Epoxidised Rapeseed Oil

Tungsten powder (4.95 g), 30% aqueous hydrogen peroxide (40 ml) and water (40 ml) were heated at 50° C. for 30 minutes with stirring. Orthophosphoric acid (2.95 g) dissolved in water (20 ml) was then added dropwise to the mixture which was stirred at 50° C. for a further 15 minutes. This prepared catalyst solution was then added with stirring to a vessel containing rapeseed oil (1000 g), water (1500 ml), 30% aqueous hydrogen peroxide (500 ml) and Adogen 464 (7.0 g) which had been preheated to 50° C. On completion of the reaction (approximately 2 hours), the aqueous layer was removed and the epoxidised rapeseed oil was used without further purification (yield: 1050 g).

The above procedure is exemplified with reference to triolein 1, which is the main triglyceride component of rapeseed oil. In the above procedure triolein 1 is converted to epoxidised triolein 3 as shown below.

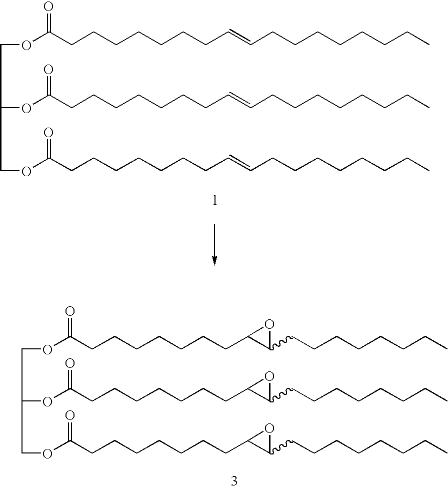

1. A glyceride compound for use in the production of a composite material, said glyceride compound comprising a first aliphatic group and a first substituent group bonded to said first aliphatic group, wherein the first substituent group has the formula —O(CH₂CH₂O)ₙP where n is 1 to 14 and P is a polymerisable group.

2. A glyceride compound according to claim 1, wherein n is 2 to 12.

3. (canceled)

4. A glyceride compound according to claim 1, wherein n is 4 to 8.

5. (canceled)

6. A glyceride compound according to claim 1, wherein the polymerisable group is a free radical addition polymerisable group.

7. (canceled)

8. A glyceride compound according to claim 1, wherein the polymerisable group is derived from substituted or unsubstituted acrylic acid.

9. A glyceride compound according to claim 8, wherein the polymerisable group is derived from methacrylic acid.

10. A glyceride compound according to claim 1, wherein the first aliphatic group contains 4 to 26 carbon atoms.

11. (canceled)

12. A glyceride compound according to claim 10, wherein the first aliphatic group contains 10 to 20 carbon atoms.

13. A glyceride compound according to claim 12, wherein the first aliphatic group contains approximately 18 carbon atoms.

14. A glyceride compound according to claim 1, wherein the first aliphatic group is saturated.

15. A glyceride compound according to claim 1, wherein a first hydroxyl group is bonded to the first aliphatic group.

16. A glyceride compound according to claim 15, wherein the first hydroxyl group is bonded to the first aliphatic group at a position which is α to the position at which the first substituent group is bonded to the first aliphatic group.

17. A glyceride compound according to claim 1, wherein the compound comprises a second aliphatic group.

18. A glyceride compound according to claim 17, wherein a second substituent group is bonded to the second aliphatic group and said second substituent group has the formula —O(CH₂CH₂O)ₘR¹, where m is 0 to 14 and R¹ is selected from the group consisting of hydrogen, alkyl and a polymerisable group.

19. A glyceride compound according to claim 17, wherein the compound comprises a third aliphatic group.

20. A glyceride compound according to claim 19, wherein a third substituent group is bonded to the third aliphatic group and said third substituent group has the formula —O(CH₂CH₂O)_{q}R², where q is 0 to 14 and R² is selected from the group consisting of hydrogen, an alkyl group and a polymerisable group.

21. A glyceride compound according to claim 1 having the formula 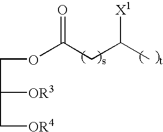
where X¹ is —O(CH₂CH₂O)ₙP, s is 0 to 23, t is 1 to 24, s+t=2 to 24, and R³ and R⁴ are each separately selected from the group consisting of hydrogen, a saturated or unsaturated aliphatic group and a group having the formula 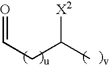
where X² is —O(CH₂CH₂O)R⁵, w is 0 to 14, R⁵ is hydrogen, an alkyl group or a polymerisable group, u is 0 to 23, v is 1 to 24 and s+t=2 to 24.

22. A glyceride compound according to claim 1 having the formula 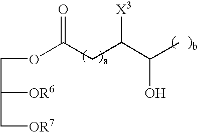
where X³ is —O(CH₂CH₂O)ₙP, a is 0 to 22, b is 1 to 23, s+t=1 to 23, and R⁶ and R⁷ are each separately selected from the group consisting of hydrogen, a saturated or unsaturated aliphatic group and a group having the formula 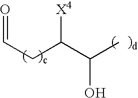
where X⁴ is —O(CH₂CH₂O)_{y}R⁵, y is 0 to 14, R⁵ is hydrogen, an alkyl group or a polymerisable group, c is 0 to 22, d is 1 to 23 and c+d=1 to 23.

23. A method for producing a substituted glyceride compound according to claim 1, wherein the method comprises reacting a glyceride starting compound having an epoxide group bonded to an aliphatic group of the starting compound with a reactant compound having the formula HO(CH₂CH₂O)ₙP or a salt thereof so as to effect cleavage of the epoxide ring of the starting compound to provide said substituted glyceride compound.

24. A method according to claim 23, wherein cleavage of the epoxide ring is effected in the presence of a Lewis acid catalyst.

25. A method according to claim 24, wherein the catalyst comprises boron trifluoride.

26-27. (canceled)

28. A method according to claim 24, wherein the glyceride starting compound and the reactant compound are dissolved in a first organic solvent prior to addition of the catalyst.

29-31. (canceled)

32. A method according to claim 28, wherein the substituted glyceride compound is separated from the catalyst by adding water to induce a phase separation process whereby the substituted glyceride compound resides substantially in an organic phase and the catalyst resides substantially in an aqueous phase, followed by separation of the organic and aqueous phases.

33-34. (canceled)

35. A method according claim 23, wherein the reactant compound is polyethyleneglycol methacrylate.

36. A method according to claim 23, wherein the method comprises the addition of a further reactant compound having the formula HO(CH₂CH₂O)ₙH where e is 1 to 14.

37. A method according to claim 23, wherein cleavage of the epoxide ring is effected at room temperature.

38. A method according to claim 23, wherein the substituted glyceride compound is dispersed in water to provide an aqueous emulsion of the substituted glyceride.

39. A method according to claim 38, wherein a polymerisation inhibitor is added to the aqueous emulsion.

40-41. (canceled)

42. A polymer resin for use in the production of a composite material, said resin being formed by copolymerising glyceride monomers and monomers of at least one further polymerisable compound, each glyceride monomer being a glyceride compound according to claim 1.

43. A polymer resin according to claim 42, wherein the at least one further polymerisable monomer is polymerisable by free radical addition.

44. A polymer resin according to claim 42, wherein the at least one further polymerisable monomer is ethylenically unsaturated.

45. A polymer resin according to claim 42, wherein the at least one further polymerisable monomer is a substituted or unsubstituted acrylate compound, or a substituted or unsubstituted aromatic compound.

46. (canceled)

47. A polymer resin according to claim 45, wherein the acrylate compound is acrylic acid.

48. A polymer resin according to claim 45, wherein the aromatic compound is styrene.

49. (canceled)

50. A method for producing a polymer resin according to claim 42, comprising copolymerising glyceride monomers and monomers of at least one further polymerisable compound, each glyceride monomer being a glyceride compound according to claim 1.

51. A method according to claim 50, wherein the glyceride monomers to be copolymerised are provided in the form of an emulsion.

52. A method according to claim 51, wherein the emulsion comprises the glyceride monomers dispersed in an aqueous medium.

53-54. (canceled)

55. A method according to claim 50, wherein the copolymerisation reaction is effected at a temperature in the range 20 to 100° C.

56. A method according to claim 50, wherein the resin is formed by copolymerising said glyceride monomers with monomers of different first and second polymerisable compounds.

57. A method according to claim 56, wherein the monomers of the first and second polymerisable compounds are mixed prior to reaction with the glyceride monomers.

58. (canceled)

59. A composite material comprising a base material and a crosslinked polymer resin formed by crosslinking the polymer resin according to claim 42.

60. A composite material according to claim 59, wherein the base material is an organic material or a ceramic material.

61. A composite material according to claim 60, wherein the organic material is selected from the group consisting of wood fibre, straw, and particulate or fibrous biomass material.

62. A composite material according to claim 60, wherein the ceramic material is sand.

63. (canceled)

64. A method for producing a composite material according to claim 59, the method comprising copolymerising glyceride monomers and monomers of at least one further polymerisable compound to provide a polymer resin according to claim 60, mixing the base material and polymer resin, crosslinking the polymer resin, and forming the base material and resin mixture into the composite material.

65. A method according to claim 64, wherein the method further comprises blending the polymer resin with a thermally activated crosslinking agent prior to mixing the polymer resin with the base material.

66. A method according to claim 65, wherein the crosslinking agent effects crosslinking of the polymer resin by reacting with hydroxyl groups present in the polymer resin.

67. A method according to claim 65, wherein the crosslinking agent effects crosslinking of the polymer resin by reacting with carboxylic acid groups present in the polymer resin.

68. A method according to claim 65, wherein an emulsion of the polymer resin is blended with the crosslinking agent at a ratio of up to 10% of the solids content of the polymer resin emulsion.

69. (canceled)

70. A method according to claim 64, wherein the activation temperature of the crosslinking agent is in the range 130° C. to 200° C.

71. A method according to claim 64, wherein the base material and the polymer resin are mixed to provide a resin loading of 5% to 25% by dry weight of the base material.

72. A method according to claim 64, wherein the base material and polymer resin mixture is dried to remove excess moisture prior to crosslinking of the polymer resin.

73. A method according to claim 72, wherein the base material and polymer resin mixture has a water content of less than 3% by weight after drying.

74. A method according to claim 64, wherein the base material and polymer resin mixture is formed in to the composite material by at least one of moulding, extruding and pressing the mixture.

75. A method according to claim 74, wherein forming of the mixture into the composite material is effected at a temperature which is sufficient to activate the crosslinking agent.

76. (canceled)

77. A glyceride compound for use as a surface active agent, said glyceride compound comprising a first aliphatic group and a first substituent group bonded to said first aliphatic group, wherein the first substituent group has the formula
—O(CH₂CH₂O)_{f}Z where f is 1 to 14 and Z is selected from the group consisting of hydrogen, an alkyl group, a sulphite group and a phosphite group.

78-95. (canceled)

96. A method for producing a substituted glyceride compound for use as a surface active agent comprising a first aliphatic group and a first substituent group bonded to said first aliphatic group in which the substituent group has the formula —O(CH₂CH₂O)_{f}Z where f is 1 to 14 and Z is selected from the group consisting of hydrogen, an alkyl group, a sulphite group and a phosphite group, wherein the method comprises reacting a glyceride starting compound having an epoxide group bonded to an aliphatic group of the starting compound with a reactant compound having the formula HO(CH₂CH₂O)_{f}Z or a salt thereof so as to effect cleavage of the epoxide ring of the starting compound to provide said substituted glyceride compound.

97-109. (canceled)
